(19) United States
(12) Patent Application Publication
(10) Pub. No.: US 2024/0301586 A1
(43) Pub. Date: Sep. 12, 2024

(54) A METHOD FOR IDENTIFYING PEPTIDE THERAPEUTICS TO TREAT A VARIETY OF CONDITIONS

(71) Applicant: Hope Patents, LLC, Santa Fe, NM (US)

(72) Inventors: Stuart Kauffman, Santa Fe, NM (US); Sui Huang, Seattle, WA (US)

(21) Appl. No.: 18/261,916

(22) PCT Filed: Jan. 18, 2022
(86) PCT No.: PCT/US2022/070221

§ 371 Date: Jul. 18, 2023

Related U.S. Application Data
(60) Provisional application No. 63/158,792, filed on Mar. 9, 2021.
(60) Provisional application No. 63/139,258, filed on Jan. 19, 2021.

(51) Int. Cl. C40B 30/06; C12N 9/22; C12N 15/10; C12N 15/86; C40B 40/02; C40B 40/08; G01N 33/50

(52) U.S. Cl. CPC C40B 30/06; C12N 9/22; C12N 15/1079; C12N 15/1086; C12N 15/86; C40B 40/02; C40B 40/08; G01N 33/5008; C12N 2740/15043; G01N 2500/10

(57) ABSTRACT

The present disclosure provides methods for identifying peptide therapeutics to treat a variety of conditions. In some embodiments, a method for identifying peptide in therapeutics to treat viral infection condition is described. In some embodiments, a method for identifying peptide therapeutics to treat cancer is described. Some embodiments related to the peptides identified by the method described herein.

### CROSS REFERENCE TO RELATED APPLICATIONS

This application claims the benefit of U.S. Provisional Application Nos. 63/139,258, filed Jan. 19, 2021, and 63/158,792, filed Mar. 9, 2021, both of which are incorporated herein by reference in their entirety.

### BACKGROUND

### Field of the Invention

The present technology generally relates to identify peptide therapeutics.

### Description of the Related Art

The COVID-19 pandemic has ravaged the world. Despite unprecedented effort at multiple fronts of drug development, currently only one antiviral agent, the small-molecule drug Remdesivir, has shown some (limited) clinical efficacy. Overall, current strategies, including the rational design seeking to block critical viral enzymes based on molecular structure, and/or large-scale screening of drugs with such activity, or attempts to modulate pathogenic mechanisms in the host using existing drugs with known molecular targets (drug repurposing) have largely failed to identify compounds with meaningful beneficial effect to control COVID-19 in the clinic.

A major reason for the limited biological activity of small molecule drugs is that such drugs have inherently little molecular leverage for impacting the activity of any protein. Small molecule compounds work best when they fit into folding pockets of active sites of proteins, typically enzymes. By contrast, they are barely capable of disrupting protein-protein interactions (PPI)—the key-lock perfect fit molecular interaction that play ubiquitous critical roles in diseases mechanism, including in microbe-host interactions through which infectious agents exert their virulence.

Most peptide drugs have been rationally designed based on structural considerations to block the activity of particular proteins, or even of PPIs in order to disrupt protein complexes. Unlike small molecule drugs, peptides have been screened largely in vitro to identify peptides that bind to a particular protein (complex), including binding to microbial antigens. Beyond binding assays and structure-guided design, peptide libraries have not been routinely subjected to functional (phenotypic) screening, i.e. in bioassays for detecting complex biological activities, such as inhibition of viral replication in cell cultures or protection of host cells. By contrast, natural peptides with antiviral or antimicrobial activity have been found in the animal kingdom, such as Mucroporin-M1, isolated from scorpion venom, or θ-defensin-1, found in macaques. See Mahendran A. Karnan, et. al., The Potential of Antiviral Peptides as COVID-19 Therapeutics, Frontiers in Pharmacology, 2020, vol. 11, p. 1475; Christine L. Wohlford-Lenane, et. al., Rhesus Theta-Defensin Prevents Death in a Mouse Model of Severe Acute Respiratory Syndrome Coronavirus Pulmonary Disease, Journal of Virology, 2009, 83 (21) 11385-11390;

Historically, bacteriophage display libraries, in which phages (viruses that infect bacterial) encode and express on their surface a random peptide, have been used to identify peptides with the ability to bind to a particular target protein in solution or on cell surfaces. Phages do not grow in mammalian cell cultures and thus, phage display libraries are not suited for phenotypic screening in mammalian cells.

Thus, there is a need for new approaches to identify peptide-based therapeutics.

### SUMMARY

Some embodiments relate to a method for identifying bioactive peptides that confer assay cells a desired phenotype. The method comprises the following steps:
(a) generating a DNA library of DNA sequences encoding a pool of peptides,
(b) introducing the DNA sequences into assay cells to express the pool of peptides,
(c) optionally applying exogenous selection pressure to the assay cells,
(d) selecting the assay cells that manifest the desired phenotype, and
(e) identifying peptides from the pool of peptides that confer the assay cells the desired phenotype by sequencing DNA isolated from the selected assay cells.

Some embodiments relate to a method for identifying bioactive peptides that confer assay cells a desired phenotype. The method comprising of the steps:
a. generating a library of DNA sequences encoding a pool of peptides that are 5-20 amino acid long,
b. placing the library of DNA sequences into a pool of plasmids,
c. constructing virus vectors for transfecting or transducing assay cells with the pool of plasmids while minimizing the loss of diversity,
d. transducing or transfecting the virus vectors to assay cells that can exhibit a desired effect to be conferred to by a peptide of the pool of peptides,
e. selecting, either via natural selection or via physical sorting of the cells, those assay cells that manifest a desired phenotype, and
f. identifying the peptides that confer the assay cells the desired phenotype by performing targeted sequencing on DNA isolated from the selected cells.

Some embodiments relate to a method for identifying bioactive peptides that confer assay cells a desired phenotype. The method comprising of the steps:
a. generating a library of DNA sequences encoding a pool of peptides that are 5-20 amino acid long,
b. placing the library of DNA sequences into a pool of plasmids,
c. constructing virus vectors for transfecting or transducing assay cells with the pool of plasmids while minimizing the loss of diversity,
d. transducing or transfecting the virus vectors to a plurality of cells,
e. separately combining the one or more of the plurality of cells with one or more other cells not transduced or transfected with the virus vectors to form a plurality of discrete microcultures, organoids, artificial organs, or microdroplet cultures,
f. selecting those microcultures, organoids, artificial organs, or microdroplet cultures that exhibit one or more desired phenotypes, and
g. identifying the peptides that confer the desired phenotype by performing targeted sequencing on DNA isolated from the selected microcultures, organoids, artificial organs, or microdroplet cultures.

Some embodiments relate to a peptide identified by the methods described herein. Some embodiments relate to a peptide comprising a peptide sequence that is 90% identical to the peptide identified by the methods described herein. Some embodiments relate to a composition comprising the combination of 2 or more peptides identified by the method described herein.

These and other features, aspects, and advantages of the present invention will become better understood with reference to the following description and appended claims.

### BRIEF DESCRIPTION OF THE DRAWINGS

FIG. 1 is a flow chart that illustrates the general screening scheme.

FIG. 2 illustrates an embodiment of a method for identifying bioactive peptides that confer assay cells the desired phenotype of resistance to SARS-COV-2. FIG. 2a shows DNA sequences encoding random peptide library of 109 sequences or more are synthesized. FIG. 2b shows plasmids for expression of random peptides are constructed. FIG. 2c shows viral vectors are generated. FIG. 2d shows target cells are transduced with the viral vectors. FIG. 2e shows the target cells are exposed to SARS-COV-2 virus. FIG. 2f shows cells with antiviral peptides survive the virus infection and targeted sequencing is performed on the DNA sequences encoding such antiviral peptides; the antiviral peptide can then be synthesized for drug lead development.

### DETAILED DESCRIPTION

Described herein is a screening approach for identifying peptide therapeutics to treat a variety of conditions. The approach utilizes a phenotypic screening scheme whereby test peptides are expressed in the same cells that manifest the desired phenotype. The phenotypic screening scheme exploits the genetic encoding and biosynthesis of peptides by the very same cells that manifest the desired phenotype that a new drug should elicit. As one non-limiting example, the desired phenotype may be cells (e.g., airway epithelial cells) that inhibit replication of a pathogenic virus (e.g., SARS-COV-2). In various embodiments, a library of random peptides generated in a novel protocol that substantially increases diversity over that achieved by current methods is screened. Genetic sequences encoding the random peptides are introduced via expression vectors into target cells where they are expressed. Depending on the target disease state, the cells may then be exposed to a disease-causing stimulus (e.g., a pathogenic virus). Cells exhibiting the desired phenotype (e.g., survival) may then be analyzed to determine the genetic sequence of the random peptide(s) that elicited that phenotype. Further screening may be conducted to validate these peptide hits, which may then advance to further studies for direct therapeutic use, or serve as leads for further optimization and identification of therapeutics.

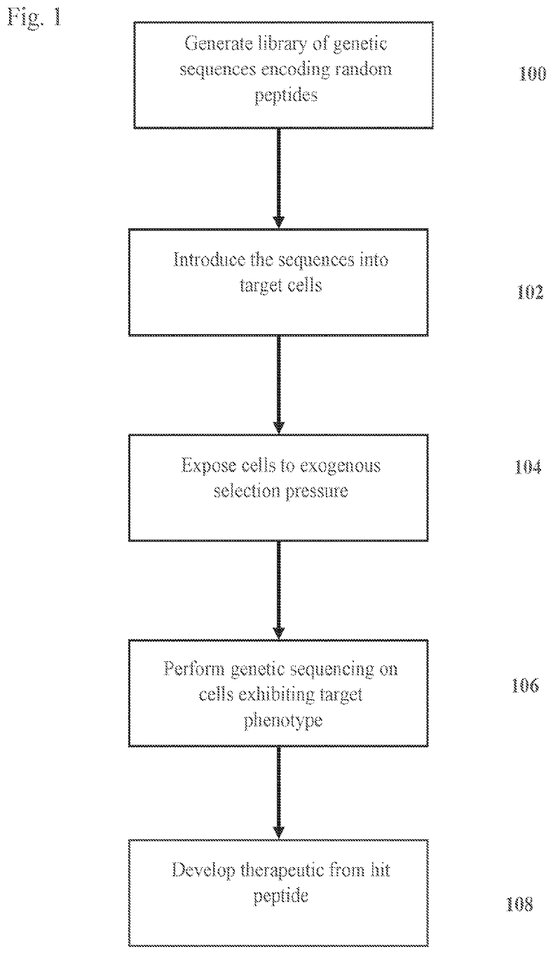

FIG. 1 is a flow chart that illustrates the general screening scheme. At step 100, a library of genetic sequences is generated that encode random peptide sequences. At step 102, the genetic sequences are introduced into the target cells. Each sequence of a random peptide is then expressed in individual or small clades of target cells. At step 104, the cells are optionally exposed to an exogenous selection pressure, such as a disease-causing stimulus. Optionally, if the cells are already in the desired screening state (e.g., if they are cancer cells), step 104 is not required. The procedure can also be used to enrich for cells that made a secreted form of the random peptide that has ability to influence other cells in the same microculture in a desired manner. At step 106, the cells exhibiting the desired phenotype are then subjected to genetic sequencing to determine which random peptides elicit the desired response. At step 108, these hit peptides may then either be directly developed into a therapeutic or serve as a lead for further therapeutic development. These steps will next be described in more detail.

### Generation of a DNA Library Encoding Random Peptide Sequences

In some embodiments, a DNA library of DNA sequences encoding a pool of peptides are generated. In some embodiments, the DNA sequences in the DNA library are randomly generated. Non-limiting methods for generating a random DNA library are described in U.S. Pat. No. 5,723,323, which is incorporated herein by reference in its entirety. The DNA sequences can be generated by known oligo synthesis techniques. The DNA sequences can then be integrated in plasmids containing a protein expression cassette, which contains elements for driving the expression of the DNA sequences. The elements for driving expression can include, for example, target cell-adjusted enhancers and promoters, transcribed but untranslated regions (UTR) flanking the coding region, splice-sites, poly (A) signals, etc.

In some embodiments, the peptides encoded by the DNA sequences are 5-20 amino acids long. In some embodiments, the peptides are 12-15 amino acids long. In still other embodiments, the peptides are 6-9 amino acids long.

In some embodiments, the randomness of the DNA sequences is constrained by probabilistic bias or a deterministic modification in amino acid sequence composition that the DNA sequences encode. In some embodiments, the DNA sequences are designed to encode peptides that satisfy desired physico-chemical properties. For example, in some embodiments, the algorithm described in Smialowski, P., Doose, G., Torkler, P., Kaufmann, S. & Frishman, D. PROSO II—a new method for protein solubility prediction. FEBS J 279, 2192-2200 (2012), which is incorporated herein by reference in its entirety, may be used to design sequences encoding for peptides that are likely to be soluble. In some embodiments, the DNA sequences are designed to encode peptides that mimic CDRs (Complementarity-determining regions) of antibodies. For example, the techniques described in Sachdeva, S., Joo, H., Tsai, J., Jasti, B. & Li, X. A Rational Approach for Creating Peptides Mimicking Antibody Binding. Scientific reports 9, 997 (2019), which is incorporated herein by reference in its entirety may be utilized. In some embodiments, the DNA sequences are designed to encode peptides that consider genetic code and codon usage, for example, by referencing codon usage tables. In some embodiments, the DNA sequences are designed to encode peptides that contain receptor binding motifs.

In some embodiments, the DNA sequences are designed to encode peptides that possess certain properties, such as stability, cell-permeability, or cell secretion.

In some embodiments, the randomly generated DNA sequences are fused to other DNA sequences encoding pre-determined peptide sequence(s) that confer a particular functionality. The particular functionality may include, but is not limited to, a cell-penetrating peptide (CPP), flanking sequences from intein for circularization, and a peptide to allow the random peptides to be secreted (i.e., a signaling peptide). See Peng C. Shi C, Cao X. Li Y, Liu F, Lu F. Factors Influencing Recombinant Protein Secretion Efficiency in Gram-Positive Bacteria: Signal Peptide and Beyond. Front Bioeng Biotechnol. 2019; 7:139, which is incorporated herein by reference in its entirety.

Cell-penetrating peptides (CPPs) are short peptides that facilitate cellular intake and uptake of molecules ranging from nanosize particles to small chemical compounds to large fragments of DNA. The “cargo” is associated with the peptides either through chemical linkage via covalent bonds (i.e., bio-synthesized as fusion peptide) or through non-covalent interactions. CPPs that may be utilized with the random peptides described herein include, but are not limited to, those described in Agrawal, P. et al. CPPsite 2.0: a repository of experimentally validated cell-penetrating peptides. Nucleic acids research 44, D1098-1103 (2016), which is incorporated herein by reference in its entirety. In some embodiments, cyclic CPP sequences are used, such as those described in Park, S. E., Sajid, M. I., Parang, K. & Tiwari, R. K. Cyclic Cell-Penetrating Peptides as Efficient Intracellular Drug Delivery Tools. Mol Pharm 16, 3727-3743 (2019), which is incorporated herein by reference in its entirety.

In some embodiments, the cell-penetrating peptide (CPP) is a HIV TAT protein or a protein encoded by Antennaepedia gene, such as described in D Derossi, S Calvet. A Trembleau, A Brunissen, G Chassaing, A Prochiantz, Cell internalization of the third helix of the Antennapedia homeodomain is receptor-independent, J Biol Chem. 1996 Jul. 26;271(30):18188-93, which is incorporated herein by reference in its entirety.

In some embodiments, flanking sequences from intein are incorporated to induce circulization of the random peptides, such as those described in Delivoria, D. C. et al. Bacterial production and direct functional screening of expanded molecular libraries for discovering inhibitors of protein aggregation. Sci Adv 5, caax5108 (2019), which is incorporated herein by reference in its entirety.

Additional sequences that may be added to, or designed into, the random sequences in order to achieve desired properties are described in Lec, A. C., Harris, J. L., Khanna, K. K. & Hong, J. H. A Comprehensive Review on Current Advances in Peptide Drug Development and Design. Int J Mol Sci 20 (2019), which is incorporated herein by reference in its entirety.

In some cases, the diversity of the library of random sequences is statistically decreased due to the skewed distribution caused by amplification steps. In some embodiments, this decrease in diversity is counteracted by exploiting their faster hybridization kinetics. In some embodiments, approaching the theoretical maximum of diversity of sequences, which is often lost due to uneven amplification steps, is achieved by a process comprised of the following steps:
(a) denaturizing and rehybridizing the DNA containing the random sequences,
(b) monitoring reannealing by spectroscopy, e.g., UV 260 nm absorption,
(c) selectively digesting some of the random sequences with heat-labile double strand-specific nuclease at a pre-determined degree of reannealing, and
(d) inactivating the nuclease by heating and continue reannealing the less abundant species.

Further details regarding this procedure is described in Peterson, D. G., Wessler, S. R. & Paterson, A. H. Efficient capture of unique sequences from eukaryotic genomes. Trends Genet 18, 547-550 (2002), which is incorporated herein by reference in its entirety.

In some embodiments, a loss of diversity of sequences is reduced by conducting in vitro recombination and mutagenesis of the DNA sequences generated as described above.

Introduction of DNA Sequences into Assay Cells

In some embodiments, introducing the DNA sequences into the assay cells comprise a method selected from transformation, transduction, and transfection.

In some embodiments, one or more DNA sequences are placed into one or more expression cassettes in plasmids before introducing into assay cells. In some embodiments, the plasmids are episomal plasmids. In some embodiments, more than one randomly generated DNA sequence may be placed into the same expression cassette in order to probe for synergistic combinations of peptides.

In some embodiments, the plasmids are introduced into virus vectors before introducing the DNA sequences into assay cells. The virus vector can be Lentivirus Adenovirus, or Baculvoirus vector. In some embodiments, the virus vectors are tagged with fluorescence so selection of virus-infected cells can be done by FACS.

In some embodiments, the DNA sequences are integrated into the genome of the cells. In some embodiments, the integration is through nuclease-mediated site-specific integration, transposon-mediated gene delivery, or virus-mediate gene delivery. In some embodiments, the nuclease-mediated site-specific integration is through CRISPR/Cas9 RNP. In some embodiments, the virus-mediated gene delivery uses lentivirus. In some embodiments, the lentivirus are generated by transfecting HEK 293T cells with the generated plasmids (for example, using the Lenti-X™ 293T lentivirus production platform).

In some embodiments utilizing viral vectors, the loss of diversity of sequences is reduced by iteratively transfecting the virus-producing cells with different samples from the plasmid pool.

In embodiments where the target assay cells are bacterial cells, the DNA sequences may be introduced into the bacterial cells using a known phage technique. For example, in some embodiments the lambda gt11 phage may be used, as is described in I. M. Chiu and K. Lehtoma, Direct cloning of cDNA inserts from lambda gt11 phage DNA into a plasmid vector by a novel and simple method, Genet Anal Tech Appl, 1990 February;7(1):18-23, which is incorporated herein by reference in its entirety.

### Selection of Assay Cells

In various embodiments, the target assay cells may be animal or bacterial cells. In some embodiments, the target assay cells are mammalian cells. In some embodiments, the target assay cells are human cells. In some embodiments, the target assay cells are selected from human airway cells, human cancer cells, in which a phenotype change can be monitored, such as differentiation of cancer stem cells into postmitotic cells.

In some embodiments, the assay cells are human airway cells and the exogenous selection pressure is virus infection. In some embodiments, the virus is SARS-Cov2. In some embodiments, the desired phenotype that the assay cells manifest is to survive after SARS-Cov2 infection. In some embodiments, the virus can be tagged with fluorescence so infection frequency or fraction of infected cells can be measured. In some embodiments, FACS can be used to select infected cells. In some embodiments, assay cells that survive are naturally enriched in the cell population, providing for self-selection of cells that exhibit the desired phenotype and contain hit peptides.

In some embodiments, the assay cells are cancer cells and the desired phenotype that the assay cells have is cell death or switching on of particular marker genes that indicated conversion to differentiated cells as originally proposed by Stuart Kauffman in the paper S. Kauffman. S. Differentiation of malignant to benign cells. J. Theor. Biol. 31, 429-451 (1971).

In some embodiments, the assay cells are microbial cells and the desired phenotype that the assay cells have is cell death. For example, the microbial cells may be bacterial cells or fungal cells.

In various embodiments, the desired phenotype that the peptide elicit in the assay cells have include, but are not limited to:
a. protecting cells from infection against a virus;
b. microbial death or growth inhibition;
c. inducing a state transition in mammalian cells, including
   i. entry of an undesired state (cancerous, inflammatory, etc.) into apoptotic path cell death,
   ii. differentiation from a cancer stem-cell like state to a postmitotic state,
   iii. differentiation from one cell type to one or more other cell types; and
d. altering the cell morphology in a specified manner.

In some embodiments, the desired phenotype is an alteration in cell population structure in a desired manner. For example, the ratio of cell phenotype in a particular population of cells may be altered into a different desired ratio.

As noted above, in some embodiments, the selection of the assay cells that exhibit the desired phenotype happens naturally via enrichment of cells exhibiting the desired phenotype (e.g., cell survival). Thus, in these embodiments, no further cell selection is required. For example, in the case of infection with SARS-COV-2 or other virus, the virus replicates and kills cells, and propagate in the cell population except in the few cells that carry the transgene that expresses a version of the peptide that confers antiviral activity. These cells will resist the virus, stay alive and continue to proliferate. Their fraction increasingly dominate the surviving cell population. The respective gene sequence of their antiviral peptides (“hits”), are also enriched.

In other embodiments, the method to select the assay cells that manifest the desired phenotype include FACS, biopanning, magnetic-bead cell separation or other cell sorting techniques. For example, in embodiments utilizing viral infection (e.g., SARS-COV-2), the virus may be tagged with fluorescence. In other embodiments, the lentivirus vector may be tagged with fluorescence. In some embodiments, the assay cells are labeled with fluorescent reporter proteins for monitoring cell viability and viral infection. In some embodiments, these techniques may be used in combination with each other and with the natural cell selection noted above.

In some embodiments, the assay described above is modified to identify peptides that cause cells to induce some change in other cells. Thus, in some embodiments, the desired property is exhibited by cells other than the cells that express the random peptides. In some embodiments, these methods comprise of the steps:
a. generating a library of DNA sequences encoding a pool of peptides that are 5-20 amino acid long,
b. placing the library of DNA sequences into a pool of plasmids,
c. constructing virus vectors for transfecting or transducing assay cells with the pool of plasmids while minimizing the loss of diversity,
d. transducing or transfecting the virus vectors to a plurality of cells,
e. separately combining the one or more of the plurality of cells with one or more other cells not transduced or transfected with the virus vectors to form a plurality of discrete microcultures, organoids, artificial organs, or microdroplet cultures,
f. selecting those microcultures, organoids, artificial organs, or microdroplet cultures that exhibit one or more desired phenotypes, and
g. identifying the peptides that confer the desired phenotype by performing targeted sequencing on DNA isolated from the selected microcultures, organoids, artificial organs, or microdroplet cultures.

In these embodiments, the random-peptide is made to be secreted by fusion to a signal peptide, the unit of selection is not an individual cell that carries the gene for the random peptide, but entire cell populations in microcultures (e.g. wells in multi-well cell culture plates, cell-culture droplets), organoids, or artificial organs. Herein, only a small fraction of the cells in the cell population contains the gene that expresses the random peptide, and their capacity to rescue the entire cell population is the target phenotype for the screening.

In some embodiments, once hit peptides are identified in the microcultures, organoids, artificial organs, or microdroplet cultures, any combinatorial subset of the hit peptides may then be screened for the desired properties.

### Identification of Hit Peptides

Once cells exhibiting the desired phenotype are isolated or enriched, they may then be analyzed to identify the sequences of the hit peptides that induced the desired phenotype. In some embodiments, the sequences are identified using DNA sequencing.

In some embodiments, the method of sequencing the DNA is next-generation gene sequencing technology, including but not limited to, targeted sequencing (e.g., utilizing flanking sequences introduced into the randomly generated sequences, as discussed above). Discovery of peptide(s) of interest (“hits”) occurs in the bulk human cell population using next-generation sequencing; this obviates the need for high-throughput parallel micro-cell culture assays as is the case with small molecule drug screening.

In some cases, random libraries generated as described above may suffer from the “unseen species” problem. Specifically, any particular sample of the library used in the assay procedure described herein may not include many of the sequences within sequence space generated. Aspects of this problem are described in Raghunathan, A., Valiant, G. & Zou, J. Estimating the unseen from multiple populations. Proceedings of the 34 th International Conference on Machine Learning, Sydney, Australia 70, (2017), which is incorporated herein by reference in its entirety. To address this issue, in some embodiments, the assay is conducted iteratively or in a massively parallel fashion. By performing multiple runs of the assay using different samples from the randomly generated library, more of the sequence space will be screened. Thus, for example, the plasmid library generated as described above may be algorithmically sampled and analyzed to cover more sequence space in addition to aforementioned prevention of loss of diversity by removal of the most abundant sequences by self-hybridization kinetics. The sampling optimization is constrained by the maximal number of clones that can be dealt with given by the number of viral vector producing cells, and can be determined by simulation and separately empirically verified by sequencing, guided by the Corbet's Rule, as described in Fisher, R. A., Corbet, A. S. & Williams, C. B. The relation between the number of species and the number of individuals in a random sample of an animal population. J Animal Ecology 12, 42-58 (1943), which is incorporated herein by reference in its entirety.

### Lead Optimization of Hit Peptides

Once hit peptides have been identified using the procedures described above, they may be utilized as a starting point to identify further peptides with increased potency for eliciting the desired phenotype. In some embodiments, multiple rounds of screening are conducted to drive evolutionary selection for more potent peptides. In some embodiments, genetic recombination of two or more identified peptides is used to generate a recombined novel random peptide library, which is then screened for efficacy. In some embodiments, where the identified peptides are secreted peptides, two or more identified peptides are combined to be screened for efficacy. In some embodiments, the hit peptides used for recombination are selected to be a set of hits that exhibit sequence similarity, which may suggest the set of hits bind to the same cellular target. In some embodiments, hit peptides used for recombination are selected to have greater than 80%, 85%, 90%, 95%, 98%, or 99% homology. In various embodiments, 2, 3, 4, 5, 6, 7, or more hit peptides are selected for recombination.

In another embodiment, diverse hit peptides are selected for recombination, suggesting they may bind different cellular targets. Thus, for example hit peptides are selected having less than 50%, 70%, 80%, 85%, 90%, 95%, 98%, or 99% homology and then subjected to recombination to generate a new library of peptides for screening. In various embodiments, 2, 3, 4, 5, 6, 7, or more hit peptides are selected for recombination.

In other embodiments, point mutations are introduced into hit peptides to generate a set of similar peptides. For example, mutations may be introduced at 1, 2, or 3 hamming distance away in sequence space to generate a “quasi-species” population. This new population of sequences may then be screened using the techniques described herein. Utilizing point mutations may utilized in addition to and after the recombination technique described above.

In some embodiments, the mutation approaches described above may be conducted iteratively to identify sequences of higher and higher efficacy. Iterations may be repeated until a sufficient level of effectiveness, or no improvement in efficacy, is reached.

Other known techniques may be used to further develop a hit peptide into a therapeutic. In some embodiments, chemical modification of the hit peptide is conducted to alter the physicochemical and pharmacokinetic properties of the peptide. For example, in embodiments where a CPP sequence had not already been incorporated into the random sequence as discussed above, the CPP sequence may be added to facilitate cellular entry when in therapeutic use. In other embodiments, designed variations to the peptide sequence are made to achieve desired properties, such as desired physicochemical properties or circularization of the peptide. In some embodiments, one or more amino acids in the sequence are changed to a D-amino acid to increase in vivo stability.

Other embodiments include incorporating an identified therapeutic peptide into a formulation package for delivery, such as incorporating into liposome or other lipid nanoparticle.

Some embodiments relate to a peptide identified by any method described herein. Some embodiments relate to a peptide comprising the combination of 2 or more peptides identified by the method described herein. Some embodiments relate to a peptide comprising a peptide sequence that is 90% identical to the peptide described herein.

In some embodiments, the hit peptides are further screened. In some embodiments, the hit peptides are made synthetically. The synthetic hit peptides can then be introduced into cells to test whether they can render the cells the desired phenotypes.

### Selection of Hit Peptides Effective Against Viral Mutation

As discussed above, some embodiments include identification of peptides effective as antivirals. In some embodiments, a collection of hit peptides exhibiting antiviral properties that are identified as discussed above are further screened to identify a subset of hits, or combination of hits, that are most effective against mutated viruses. Such subset of hits, or combination of hits, may then be used against a particular circulating virus, with an expectation that the subset or combination will continue to be effective against mutated strains of the virus. Such a therapy would also be expected to inhibit the accumulation of viral mutations within hosts that have been treated with the therapy.

In some embodiments, the additional screen comprises infecting all or a fraction of cells within a human cell culture with a lentivirus expressing a randomly chosen single or combination of hit peptides identified as described above. In some embodiments, one or more lentivirus is used that express 2, 3, 4, 5, 6, 7, or more hit peptides. In some embodiments, the fraction of cells are generated by infecting a first collection of cells followed by addition of non-infected cells. After a sufficient growth period (e.g. 6 hours), the cells are infected with the target virus (e.g., SARS-COV2). In some embodiments, the cells and/or the target virus may be labeled to permit monitoring of infection. For example, the cells may be labeled with green fluorescent protein and the virus may be labeled with flourescene. The ratio of cell signal to virus signal allows testing of the capacity of hit peptide(s) to protect infected cells. The accumulation of mutations in the virus may be monitored by sequencing the viruses (e.g., using targeted or deep sequencing) at various (e.g., pre-determined) time points.

In one variation, fractions of the initial cell culture are transferred to different culture containers, and this process may be repeated with serial transfer of fractions of cells. In some embodiments, the number of cells and transfer rate may be varied across the serial transfers. Viruses within each culture container, or a subset of culture containers, may be sequenced (e.g., using targeted or deep sequencing) to determine the existence of viral mutations.

Sequencing can be used to obtain the number of viral variants, which variants are still propagating and which have gone extinct, the number of copies of each variant, and the number of mutations accumulated for each variant. In some embodiments, these variables may be used to determine when to stop the experiment.

This experiment may be repeated with different randomly chosen hit peptides or combination of hit peptides. Those hit peptides or combination of peptides that reduce or slow the incidence of viral evolution may then be selected for development and use as a therapeutic. This process may also be used to identify likely mutations of the virus. These mutated strains could then be used in the initial hit peptide identification process described above.

In some embodiments, the results of the above experiment is used to generate a phylogenetic graph. The effectiveness of any hit peptide, combination of peptides, or concentrations of hit peptides can be assessed based on study of the phylogenetic graph. Slowing or stopping of the evolution of the virus can be indicated by a reduction in the branching structure of the phylogenetic graph. As effectiveness in slowing evolution increases, fewer branches will be found at comparable time points post infection.

In some embodiments, the results of the above experiment are used to establish the number of copies of each viral variant as these variants emerge, persist and perhaps go extinct in the course of the process.

In some embodiments, hit peptides, or combination of peptides, are chosen that are effective to 1) prevent emergence of any new strains, 2) cause any new strains to go extinct over a pre-determined time of culture and total number of cells in the system, or 3) do not permit any further new strains to emerge after a pre-determined point of time, and does not allow the initial virus to continue to propagate in the population after that point in time.

In some embodiments, combinations of hit peptides having the desired anti-mutation properties may be determined by testing all combinations and sub-combinations of a set of hit peptides using the experiments described above. The best performing combination of hit peptides may then be used for therapeutic development. In another embodiment, a combination of hit peptides may be identified by starting with a first trial set of hit peptides that is a subset of the whole set of peptides. First subsequent sets may include one addition or one subtraction of a hit peptide from the first trial set. All such first subsequent sets are tested and the best one is used as a new starting set from which a second subsequent set may be tested that include all one-addition or one-subtraction combination variants from the new starting set. This “hill climbing” process may continue until a desired level of efficacy is achieved. It will be appreciated that other hill climbing optimization algorithms may be used to identify a suitable final set of hit peptides in an efficient manner.

### Identification of Cellular Targets

Some embodiments relate to a method to identify cellular targets of peptides identified herein. This identification may thus provide new druggable targets for therapeutic intervention. Once the target is known, various methods for identifying therapeutics hitting that target may be utilized, including traditional screening or medicinal chemistry approaches. Any known suitable method for identifying the cellular target of a hit peptide may be used, including affinity based methods. In some embodiments, protein-protein interaction partners and cellular pathways involved in the mechanisms of action of the peptide are identified.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Example 1

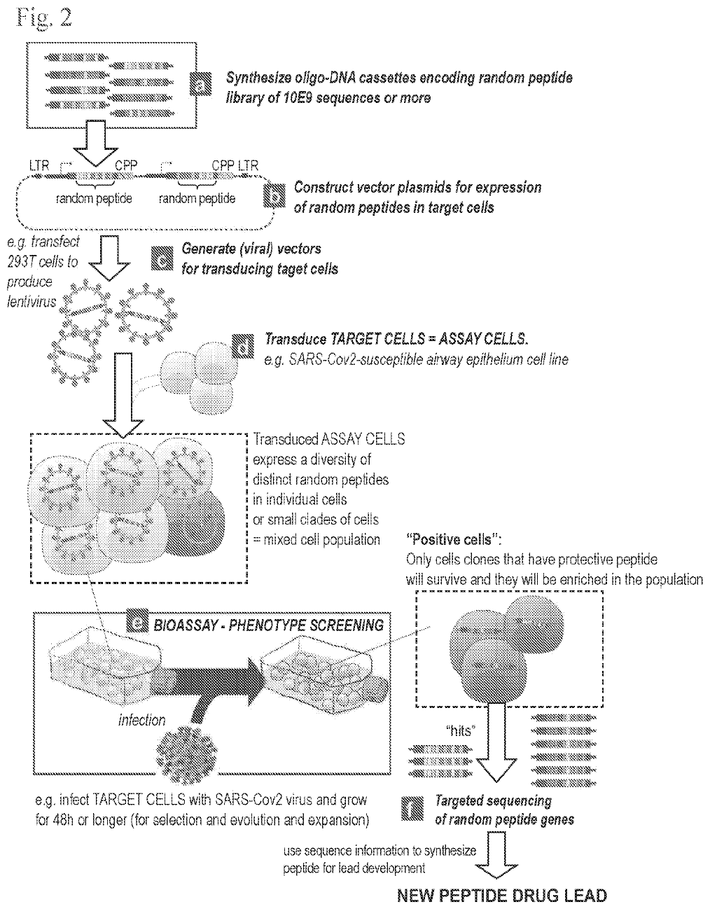

An experiment is conducted to identify peptides conferring resistance to SARS-COV-2 infection. The experiment is illustrated in FIG. 2. In FIG. 2a, DNA sequences encoding random peptide library of 109 sequences or more are synthesized. In FIG. 2b, plasmids containing protein expression cassettes are constructed. The plasmids include one or more of the expression cassettes encompassing the peptide-encoding DNA sequences that include a CPP sequence, and regulatory sites for expression in the target cell, as well as sequence elements for generating viral vectors (flanking LTR sequences, packaging signals). In FIG. 2c, lentivirus vectors are generated utilizing 293T cells. In FIG. 2d, target cells are transduced with the viral vectors. Human lung epithelial cells susceptible for SARS-Cov2 virus are transduced with lentiviral vectors that contain the gene expression cassettes encoding the random peptides, such that each cell in the cell culture (population of hundreds of millions of cells) expresses a distinct version of the random peptide. The transduced human lung epithelial cells are grown to confluence. In FIG. 2e, the target cells are exposed to SARS-Cov2 virus. The virus replicates and kills cells and propagate in the cell population except in the few cells that carry the DNA sequence that expresses a version of the peptide that confers antiviral activity. These cells will resist the virus, stay alive and continue to proliferate. Their fraction increasingly dominates the surviving cell population. The respective gene sequence of their antiviral peptides, are also enriched and are identified by nextgen DNA sequencing of the entire surviving cell culture at once. In FIG. 2f, cells with antiviral peptides survive the virus infection and targeted sequencing is performed on the DNA sequences encoding such antiviral peptides; the antiviral peptides can then be synthesized for drug lead development.

### Example 2

This example shows one application of selecting hit peptides that are effective against or that block the mutation of SARS-COV-2 virus.

The hit peptides having antiviral activity against SARS-COV-2 virus are identified according to the procedures in Example 1.

DNA sequences encoding these antiviral peptides are inserted into lentivirus vectors. Lentivirus is produced as previously described. Y. Kong, et. al., Int. J. Clin. Exp. Pathol., 2017; 10(6):6198-6209. Briefly, Lentivirus is produced by co-transfecting 293T cells with a 4-plasmid system including lentiviral expression plasmid containing the antiviral peptides and three packaging plasmids with the help of Lipofectamine™M2000. The resulting culture supernatant containing lentiviral particles are collected at 48 h and 72 h post-transfection and then the pooled supernatant is centrifuged via ultra-centrifugation for 2 h at 50,000 g (Optima L-90K. Beckman, USA). Infectious titer of the preparations are determined using inverted fluorescence microscope (Ti-s, Nikon, JPN) by detecting the percentage of GFP positive 293T cells after transfected with serial dilutions of concentrated lentivirus.

Human lung cells are transfected with the resulted lentiviruses. After a period of time to allow the transfected cells to express the antiviral peptides, the transfected cells are mixed with other human lung cells that have not been transfected in a culture flask. Then the mixed cells are infected with SARS-Cov2 virus.

The infected cells are then divided into separate flasks and the process is repeated serially. After several passages of the cells, targeted sequencing is conducted to determine evolution and propagation of the SARS-Cov2 virus. Based on these results, one or more antiviral peptides are chosen that are effective against or block viral mutation.

In the Detailed Description Section, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

It is to be understood that the presently disclosed and claimed inventive concept(s) is not limited in its application to the details of construction and the arrangement of the components set forth in the following description or illustrated in the drawings, experimentation and/or results. The presently disclosed and claimed inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and/or claimed inventive concept(s) shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo—or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2^{nd} ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

All patents, published patent applications, and non-patent publications mentioned in the specification are indicative of the level of skill of those of ordinary skill in the art to which this presently disclosed and/or claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed and/or claimed herein can be made and executed without undue experimentation in light of the present disclosure. While the compositions and methods of the inventive concept(s) have been described in terms of particular embodiments, it will be apparent to those of ordinary skill in the art that variations may be applied to the compositions and/or methods and in the steps or in the sequence of steps of the method described herein without departing from the concept, spirit and scope of the presently disclosed and/or claimed inventive concept(s). All such similar substitutes and modifications apparent to those of ordinary skill in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more.” “at least one,” and “one or more than one.” The singular forms “a,” “an,” and “the” include plural referents unless the context clearly indicates otherwise. Thus, for example, reference to “a compound” may refer to 1 or more, 2 or more, 3 or more, 4 or more or greater numbers of compounds. The term “plurality” refers to “two or more.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example but not by way of limitation, when the term “about” is utilized, the designated value may vary by +20% or +10%, or +5%, or +1%, or +0.1% from the specified value, as such variations are appropriate to perform the disclosed methods and as understood by persons having ordinary skill in the art. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

As used in this specification and claim(s), the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “cancer” denotes a malignant neoplasm that has undergone genetic, epigenetic or phenotypic alterations with loss of differentiation, increased rate of growth, invasion of surrounding tissue, and is capable of metastasis. The term “cancer” shall be taken to include a disease that is characterized by uncontrolled growth of cells within a subject. In some embodiments, the terms “cancer” and “tumor” are used interchangeably. In some embodiments, the term “tumor” refers to a benign or non-malignant growth.

Although the invention has been described with reference to embodiments and examples, it should be understood that numerous and various modifications can be made without departing from the spirit of the invention. Accordingly, the invention is limited only by the following claims.

What is claimed is:

1. A method for identifying bioactive peptides that confer assay cells a desired phenotype, the method comprising the steps of:
(f) generating a DNA library of DNA sequences encoding a pool of peptides,
(g) introducing the DNA sequences into assay cells to express the pool of peptides,
(h) optionally applying exogenous selection pressure to the assay cells,
(i) selecting the assay cells that manifest the desired phenotype, and
(j) identifying peptides from the pool of peptides that confer the assay cells the desired phenotype by sequencing DNA isolated from the selected assay cells.

2. The method of claim 1, wherein the DNA sequences in the DNA library are randomly generated.

3. The method of claim 2, wherein the randomness of the DNA sequences is constrained by probabilistic bias or a deterministic modification in amino acid sequence composition that the DNA sequences encode.

4. The method of claim 2 or 3, wherein the randomly generated DNA sequences are fused to other DNA sequences encoding pre-determined peptide sequence(s) that confer a particular functionality within one single fusion peptide, or as multiple peptides, encoded by a multi-cistronic transcripts in the same expression cassette, or in distinct expression cassettes.

5. The method of claim 1, wherein the length of the peptides in the pool is 5-20 aa long.

6. The method of claim 1, wherein introducing the DNA sequences into the assay cells comprise a method selected from transformation, transduction, and transfection.

7. The method of claim 1, wherein one or more DNA sequences are placed into one or more expression cassettes in plasmids before introducing into assay cells.

8. The method of claim 7, wherein a diversity of sequences is increased by correcting a skewed distribution of species frequency caused by amplification by using hybridization kinetics.

9. The method of claim 8, wherein the increase of diversity of sequences is achieved by:
(a) denaturizing and rehybridizing the DNA containing the random sequences,
(b) monitoring reannealing by spectroscopy,
(c) selectively digesting some of the random sequences with double strand-specific nuclease at a pre-determined degree of reannealing, and
(d) inactivating the nuclease.

10. The method of claim 7, wherein the plasmids are introduced into virus vectors before introducing the DNA sequences into assay cells.

11. The method of claim 10, wherein a loss of diversity of sequences is reduced by iterative transfection of the vector virus-producing cells.

12. The method of claim 10, wherein a loss of diversity of sequences is reduced by conducting in vitro recombination and mutagenesis of the DNA sequences.

13. The method of claim 1, wherein the DNA sequences are integrated into the genome of the cells.

14. The method of claim 13, wherein the integration is through nuclease-mediated site-specific integration, transposon-mediated gene delivery, or virus-mediate gene delivery.

15. The method of claim 14, wherein the nuclease-mediated site-specific integration is through CRISPR/Cas9 RNP.

16. The method of claim 14, wherein the virus-mediated gene delivery uses lentivirus.

17. The method of claim 1, wherein the assay cells are selected from human airway cells, cancer cells, and bacterial cells

18. The method of claim 17, wherein the assay cells are human airway cells and the exogenous selection pressure is virus infection.

19. The method of claim 18, wherein the virus is SARS-Cov2.

20. The method of claim 19, wherein the desired phenotype that the assay cells manifested is to survive after SARS-Cov2 infection.

21. The method of claim 1, wherein the assay cells are cancer cells and the desired phenotype that the assay cells have is cell death.

22. The method of claim 1, wherein the desired phenotype that the assay cells have include transition from an undesired state into a desired state.

23. The method of claim 1, wherein the method to select the assay cells that manifest the desired phenotype include natural selection or cell sorting.

24. The method of claim 1, wherein the method of sequencing the DNA is next-generation targeted gene sequencing.

25. A peptide identified by the method of any one of claims 1-24.

26. A composition comprising the combination of 2 or more peptides identified by the method of any one of claims 1-24.

27. A peptide comprising a peptide sequence that is 90% identical to the peptide of claim 25.

28. A method for identifying bioactive peptides from a pool of random peptides, the method comprising of the steps:
a. generating a library of DNA sequences encoding a pool of peptides that are 5-20 amino acid long,
b. placing the library of DNA sequences into a pool of plasmids,
c. constructing virus vectors for transfecting or transducing assay cells with the pool of plasmids while minimizing the loss of diversity,
d. transducing or transfecting the virus vectors to assay cells that can exhibit a desired effect to be conferred to by a peptide of the pool of peptides,
e. selecting, either via natural selection or via physical sorting of the cells, those assay cells that manifest a desired phenotype, and
f. identifying the peptides that confer the assay cells the desired phenotype by performing targeted sequencing on DNA isolated from the selected cells.

29. The method of claim 28, further comprising one of:
a. conducting genetic recombination of two or more of the identified peptides to generate a pool of recombined novel random peptides, and
b. introducing point mutations to an identified peptide to generate a set of similar peptides.

30. The method of claim 1 or 28, wherein the desired phenotype conferred by a peptide of the pool of peptides comprises:
a. protecting cells from infection against a virus;
b. microbial death or growth inhibition;
c. inducing a state transition in mammalian cells; or
d. altering cell morphology.

31. The method of claim 1 or 28, further comprising identifying protein-protein interaction partners or cellular pathways involved in the mechanisms of action of the identified peptides.

32. A method for identifying bioactive peptides from a pool of random peptides, the method comprising of the steps:
a. generating a library of DNA sequences encoding a pool of peptides that are 5-20 amino acid long,
b. placing the library of DNA sequences into a pool of plasmids,
c. constructing virus vectors for transfecting or transducing assay cells with the pool of plasmids while minimizing the loss of diversity,
d. transducing or transfecting the virus vectors to a plurality of cells,
e. separately combining the one or more of the plurality of cells with one or more other cells not transduced or transfected with the virus vectors to form a plurality of discrete microcultures, organoids, artificial organs, or microdroplet cultures,
f. selecting those microcultures, organoids, artificial organs, or microdroplet cultures that exhibit one or more desired phenotypes, and
g. identifying the peptides that confer the desired phenotype by performing targeted sequencing on DNA isolated from the selected microcultures, organoids, artificial organs, or microdroplet cultures.

33. The method of claim 1 or 28, wherein the desired phenotype is anti-viral effectiveness and the method further comprises:
constructing virus vectors configured to deliver DNA expressing one or more of the identified peptides;
infecting a human cell culture or portion of a human cell culture with the virus vectors expressing the one or more of the identified peptides;
growing the human cell culture for a period of time;
infecting the human cell culture or a portion of the human cell culture with the virus to which anti-viral effectiveness is desired;
conducting DNA sequencing of the virus to which anti-viral effectiveness is desired at a plurality of time points to determine the presence of viral mutations; and
selecting one or more of the identified peptides based on their ability to inhibit the development of viral mutations.
